SEQUENCE LISTING (12 sequences)

SEQ ID NO: 1 - DNA, 2004 bp, Escherichia coli
  GUS cDNA sequence
atggggttacgtcctgtagaaaccccaacccgtgaaatcaaaaaactcgacggcctgtgggcattcagtctggatagggagaactgtggaatcgaccaacgttggtgggaaagcgcgttacaagaaagccgggctatcgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtctacgtaagtttctgcttctacctttgatatatatataataattatcattaattagtagtaatataatatttcaaatatttttttcaaaataaaagaatgtagtatatagcaattccttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatataaccgtggtgacgcatgtcgcgcaagactgtaaccacgcttctgttgactggcaagttgtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatttacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggttcacgaccacgctcttatggactggattggggccaactcctaccgtacctcgcattacccttacgctgaagaaatgctcgactgggcagatgaacatggcatcgtggtgattgatgaaactgctgctgtcggctttaacctctctttaggcattggtttcgaggcgggcaacaagccgaaagaactgtacagcgaggaagcagtcaacggggaaactcagcaagcgcacttacaggcgatcaaggagctgatagcgcgtgacaaaaaccacccaagcgtggtgatgtggagtattgccaacgaaccggatacccgtccgcaaggagctagggagtatttcgcgccactggcggaagcaaccagaaaactcgacccgaccaggccgatcacctgtgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaggagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcaccagggatcgcaaaccgaagtcggcggcttttctgctgcaaaaacgctggactggcatgaacttcggtgaaaaaccgcagcagggaggcaaacaatga

SEQ ID NO: 2 - DNA, 720 bp, Aequorea victoria
  GFP cDNA sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 678 bp, Discosoma sp.
  DsRed cDNA sequence
atggggtcttccaagaatgttatcaaggagttcatgaggtttaaggttcgcatggaaggaacggtcaatgggcacgagtttgaaatagaaggcgaaggagaggggaggccatacgaaggccacaataccgtaaagcttaaggtaaccaaggggggacctttgccatttgcttgggatattttgtcaccacaatttcagtatggaagcaaggtatatgtcaagcaccctgccgacataccagactataaaaagctgtcatttcctgaaggatttaaatgggaaagggtcatgaactttgaagacggtggcgtcgttactgtaacccaggattccagtttgcaggatggctgtttcatctacaaggtcaagttcattggcgtgaactttccttccgatggacctgttatgcaaaagaagacaatgggctgggaagccagcactgagcgtttgtatcctcgtgatggcgtgttgaaaggagagattcataaggctctgaagctgaaagacggtggtcattacctagttgaattcaaaagtatttacatggcaaagaagcctgtgcagctaccagggtactactatgttgactccaaactggatataacaagccacaacgaagactatacaatcgttgagcagtatgaaagaaccgagggacgccaccatctgttcctttag

SEQ ID NO: 4 - DNA, 945 bp, Renilla
  Renilla luciferase (Rluc) cDNA sequence
atggcttcgaaggtgtacgaccccgagcagaggaagaggatgatcaccggcccccagtggtgggccaggtgcaagcagatgaacgtgctggacagcttcatcaactactacgacagcgagaagcacgccgagaacgccgtgatcttcctgcacggcaacgccgctagcagctacctgtggaggcacgtggtgccccacatcgagcccgtggccaggtgcatcatccccgatctgatcggcatgggcaagagcggcaagagcggcaacggcagctacaggctgctggaccactacaagtacctgaccgcctggttcgagctcctgaacctgcccaagaagatcatcttcgtgggccacgactggggcgcctgcctggccttccactacagctacgagcaccaggacaagatcaaggccatcgtgcacgccgagagcgtggtggacgtgatcgagagctgggacgagtggccagacatcgaggaggacatcgccctgatcaagagcgaggagggcgagaagatggtgctggagaacaacttcttcgtggagaccgtgctgcccagcgttatcatgagaaagctggagcccgaggagttcgccgcctacctggagcccttcaaggagaagggcgaggtgagaagacccaccctgagctggcccagagagatccccctggtgaagggcggcaagcccgacgtggtgcagatcgtgagaaactacaacgcctacctgagagccagcgacgacctgcccaagatgttcatcgagagcgaccccggcttcttcagcaacgccatcattgagggcgccaagaagttccccaacaccgagttcgtgaaggtgaagggcctgcacttcagccaggaggacgcccccgacgagatgggcaagtacatcaagagcttcgtggagagagtgctgaagaacgagcagagatctatctag

SEQ ID NO: 5 - DNA, 2013 bp, Arabidopsis thaliana
  ALS cDNA sequence
atggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtgcatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactga

SEQ ID NO: 6 - DNA, 1605 bp, Amaranthus tuberculatus
  PPO cDNA sequence
atggttattcagtctattacccacctctccccaaacctcgctttgccatctccactttctgtgtccaccaagaactacccagttgctgtgatgggcaacatctctgagagagaggaacctacctctgctaagagggttgcagttgttggagctggtgtttctggacttgctgctgcttacaagctcaagtcccacggactttcagtgacccttttcgaggctgattctagggctggtggaaagcttaagaccgtgaagaaggatggcttcatctgggatgagggtgctaacactatgaccgagtctgaggctgaggtgtcctcccttattgatgatcttggcctcagagagaagcaacagctcccaatctctcagaacaagcgttacattgctagggatggacttccagtgctcctcccatctaacccagctgctttgctcacctccaacatcctttccgctaagtccaagctccagatcatgctcgaaccattcctttggaggaagcacaacgctaccgagctttctgatgagcacgttcaagagtctgtgggcgagttcttcgagaggcatttcggcaaagaattcgtggactacgtgatcgatccattcgttgctggaacttgcggaggtgatcctcagtctctttctatgcatcacaccttcccagaggtgtggaacatcgagaagaggttcggatctgtgttcgctggccttatccagtccaccctcttgtccaagaaagaaaagggtggcgagaacgcctccatcaagaagccaagagttaggggctcattcagcttccaaggtggaatgcaaaccctcgtggataccatgtgcaagcagcttggagaggatgagcttaagttgcagtgcgaggtgctcagcctttcctataaccagaagggaatcccatccctcggcaactggtctgtgtcatctatgtccaacaacacctccgaggaccagtcttacgatgctgttgttgtgaccgccccaatccgtaacgtgaaagaaatgaagatcatgaagttcggcaaccccttctccctcgacttcattccagaggttacctacgtgccactctccgtgatgattaccgctttcaagaaagacaaggtgaagaggccactcgagggattcggagtgctcattccttctaaagagcagcacaacggactcaagactgagggaaccctcttctcctctatgatgttcccagatagggccccttccgatatgtgccttttcactactgttgtgggcggctctaggaacagaaagcttgctaacgcttccaccgacgagctgaagcagatcgtgtcatctgatcttcagcagcttctcggaaccgaggacgaaccatctttcgtgaaccacctcttctggtccaacgctttcccactttacggccacaactacgattctgtgctcagggctatcgacaagatggaaaaggatctccccggcttcttctacgctggaaaccataagggtggtctgtctgtgggaaaggctatggcttctggatgcaaggctgctgagcttgtgatctcctacctcgactctcacatctacgtgaagatggacgaaaagaccgcctga

SEQ ID NO: 7 - protein, 11 aa, Artificial Sequence
  BP100
KKLFKKILKYL

SEQ ID NO: 8 - protein, 9 aa, Artificial Sequence
  R9
RRRRRRRRR

SEQ ID NO: 9 - protein, 9 aa, Artificial Sequence
  D-R9 (D form of R9, synthetic)
RRRRRRRRR

SEQ ID NO: 10 - protein, 18 aa, Artificial Sequence
  KH9
KHKHKHKHKHKHKHKHKH

SEQ ID NO: 11 - protein, 29 aa, Artificial Sequence
  BP100(KH)9
KKLFKKILKYLKHKHKHKHKHKHKHKHKH

SEQ ID NO: 12 - protein, 27 aa, Artificial Sequence
  BP100CH7
KKLFKKILKYLHHCRGHTVHSHHHCIR

(19) United States
(12) Patent Application Publication
(10) Pub. No.: US 2023/0323373 A1
(43) Pub. Date: Oct. 12, 2023

(54) SPRAYABLE CELL-PENETRATING PEPTIDES FOR SUBSTANCE DELIVERY IN PLANTS

(71) Applicant: BASF Plant Science Company GmbH, Ludwigshafen (DE)

(72) Inventors: Yuki MATSUBA, Research Triangle Park, NC (US); Vinitha CARDOZA, Research Triangle Park, NC (US); Baochun LI, Morrisville, NC (US); Marianela RODRIGUEZ, Research Triangle Park, NC (US); Joerg BAUER, Research Triangle Park, NC (US); Keiji NUMATA, Saitama (JP); Jordan Corinne THERIOT, Research Triangle Park, NC (US); Chonprakun THAGUN, Saitama (JP)

(21) Appl. No.: 18/025,530

(22) PCT Filed: Sep. 7, 2021
(86) PCT No.: PCT/EP2021/074569

§ 371 Date: Mar. 9, 2023

Related U.S. Application Data
(60) Provisional application No. 63/077,186, filed on Sep. 11, 2020.

(51) Int. Cl. C12N 15/82

(52) U.S. Cl. CPC C12N 15/8216; C12N 15/8201

(57) ABSTRACT

The invention relates to a complex comprising a Cell Penetrating Peptide and one or more nucleic acids which can be applied to a plant by spraying and which can trigger a physiological outcome. Hereto, the one or more nucleic acids complex with a Cell Penetrating Peptide can be dissolved in water without the presence of additional components in the solution.

### FIELD

The present disclosure relates generally to the field of molecular biology and concerns an improved method for delivering nucleic acids into plants.

### BACKGROUND

A trait of particular economic interest is increased yield. Yield is normally defined as the measurable produce of economic value from a crop. This may be defined in terms of quantity and/or quality. Yield is directly dependent on several factors, for example, the number and size of the organs, plant architecture (for example, the number of branches), seed production, leaf senescence and more. Root development, nutrient uptake, stress tolerance and early vigour may also be important factors in determining yield. Optimizing the above-mentioned factors may therefore contribute to increasing crop yield. A further important trait is that of improved stress tolerance. Abiotic stress is a primary cause of crop loss worldwide, reducing average yields for most major crop plants by more than 50% (Wang et al., Planta 218, 1-14, 2003). Abiotic stresses may be caused by drought, salinity, extremes of temperature, chemical toxicity and oxidative stress. Biotic stresses are typically those stresses caused by pathogens (such as bacteria, viruses, and fungi), weeds, nematodes and insects, or other animals, which may result in negative effects on plant growth. The ability to improve plant tolerance to abiotic or biotic stress would be of great economic advantage to farmers worldwide and would allow for the cultivation of crops during adverse conditions and in territories where cultivation of crops may not otherwise be possible.

The ever-increasing world population and the dwindling supply of arable land available for agriculture fuels research towards increasing the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to directly modify the genomes of animals and plants.

There are numerous methods for modifying plant genomes, e.g. by crossing preferable alleles into the genome, selection of epigenetic changes, mutations induced by radiation or chemicals, chromosome duplications, genetic engineering through transfer of DNA by biolistics or Agrobacterium, transient regulation using nucleic acids (e.g. RNAi) or small molecules (e.g. salicylic acid) or regulation using different light patterns. Plants with enhanced agronomic benefits may also be generated using genome editing, improving regeneration capacity, ribonucleoparticle binding, protein inactivation or intracellular transport regulation. Several methods for genome editing have already been described.

Beside zinc-fingers, meganucleases and TALEN, CRISPR (clustered regularly interspaced short palindromic repeats) is a popular methodology for precise genome editing.

For many of these methods, there is a general need to deliver biomolecules into plant cells, in some case, the plant cells are of a specific type (e.g. regenerating plant cells, meristematic cells, root cells etc.). Current technologies have limitations, e.g. for the delivery of nucleic acids into plant cells. Two technologies are commonly used for the delivery of nucleic acids into plant cells, biolistics and Agrobacterium. The biolistics approach often suffers from low success rates, high damage of cells, and complex integration of transgenes and requires considerable experimental time and effort. Agrobacterium-mediated delivery can be really efficient, but its success is often genotype-dependent. One important point to make here is the Agrobacterium-mediated in planta transient transgene expression. It is a very effective system and widely used for assaying various kinds of gene expression systems and for protein productions, but it is only effectively working on one plant species, which is Nicotiana benthamiana.

Therefore, there is a need to develop alternative delivery systems to facilitate the development plants with enhanced agronomic benefits in fields like resistance to fungal disease, resistance to insects, germination vigor, leaf symmetry, leaf senescence, circadian rhythm, photosynthesis regulation, meristem formation, pollen formation, pollination, seed setting, seed ripening, composition of seeds, seed size, seed number and abiotic stress tolerance such as water use efficiency, nitrogen use efficiency, phosphate use efficiency, salt stress resistance, improved UV light tolerance, improved micronutrient uptake, cold tolerance or heat tolerance.

Methods using Cell Penetrating Peptides (CPPs) have become an attractive alternative for the introduction of proteins or nucleic acids into animal cells. CPPs have the ability to pass through a cellular membrane, and can thereby transport macromolecules from outside into a cell. Over the years, many CPPs have been characterised, and are classified into three groups, according to their physicochemical properties (Derakhshankhah and Jafari, Biomedicine & Pharmacotherapy 108:1090-196, 2018): cationic CPPs with polyarginine groups, amphipathic CPPs containing lysine residues, and hydrophobic CPPs having hydrophobic or non-polar motifs. An alternative classification is based on their origin (Derakhshankhah and Jafari, 2018): they can be isolated domains from larger proteins, they can be chimeric, combining domains from different peptides, or they can be synthetic. The mode of action, i.e. the way CPPs move across the cell membrane, is not resolved yet. Possible mechanisms include direct penetration of the membrane, or endocytosis.

The ways of applying CPPs on plant cells, tissues or organs for introducing a compound of interest into plant cells are limited to in-vitro methods or lab-scale methods, such as leaf infiltration (Fuime et al. 2016, Meth. Mol. Biol), fruit injection (Thagun et al. 2019 Adv. Sci.) or seed vacuum infiltration (Yoshizumi et al. 2018 Biomacromolecules). However, none of these methods are suited for high throughput or large-scale application. Moreover, the compositions for applying CPPs associated with a compound of interest and for introducing the compound of interest into plant cells are complex and require various additives. EP2974595 describes compositions that comprise chelators and surfactants, and a two-step method for applying such compositions. US 2018/0235210 discloses methods for targeting the bacterial phytopathogen Erwinia amylovora and provides compositions that include nonionic surfactants or ionic surfactants such as cationic or anionic surfactants. WO2017025967 provides methods and compositions for gene silencing by delivering a nucleic acid active in RNA pathways of plant cells; the compositions used therefore contain cell wall degrading enzymes (such as cellulases, hemicellulases, lignin-modifying enzymes, cinnamoyl ester hydrolases and pectin-degrading enzymes, alone or in combination) and a nucleic acid condensing agent (such as protamine, spermidine3+, spermine4+, hexamine cobalt, polycationic peptides such as polylysine and polyarginine, histones HI and H5 and polymers such as PEG, polyaspartate and poly glutamate). WO2015/200539 discloses compositions that must contain an osmolyte and WO2015/812530 describes a method for delivering dsRNA into a plant cell using a composition supplemented with a surfactant, salt, humectant, or a chelating agent. The method for in planta delivery comprises a drying step followed by treating the leaves with sandpaper to deliver dsRNA into the cells. WO2016/179180 describes compositions for protecting bees from pests like Varroa, which compositions comprise an anti-parasitic, anti-pest or insecticidal nucleic acid molecule, and an excipient selected from protein, pollen, carbohydrate, polymer, liquid solvent, sugar syrup, sugar solid, and semi-solid feed. However, it is known in the art that additives like chelators, surfactants, humectants or osmolytes may exert a negative influence on the stability or activity of the compound of interest that must be introduced into a plant cell.

PCT/EP2019/0866682 discloses a complex comprising a CPP and a polycation sequence, along with a second component comprising a ribonucleic acid or polynucleotide wherein the peptide is a cyclic peptide comprising at least two cysteine residues. However, the uptake of the complex is only shown for callus tissue derived from a plant.

It is an object of the present invention to simplify both the compositions containing a compound of interest complexed to CPPs, and the method of applying such compositions to plants, plant organs or to plant tissues.

### DETAILED DESCRIPTION

To overcome the limitations of the current methods for introducing biomolecules of interest into plant cells by applying Cell Penetrating Peptides (hereafter CPPs) on plants or parts thereof, the inventors have developed methods that are easy to use, on small or large scale, and under various conditions, including field conditions. The inventors further optimized the formulation of compositions for delivering biomolecules of interest through the use of CPPs.

The inventors have surprisingly found that a solution of CPPs, complexed with nucleic acids, can be applied to a plant surface by way of spraying or similar technique and that such complex can efficiently enter the plant cell. Such techniques can be used in methods like genome editing, targeted mutagenesis, transient regulation by peptides or proteins, transient regulation by RNAi and for intra-and intercellular transport of proteins/peptides in plants. Moreover, the non-invasive nature of spraying makes the method ideal for delivery without damage to the plant tissue. In addition, the inventors have surprisingly found that the nucleic acid-CPP complex to be introduced into a plant cell of a plant, can simply be dissolved in water and when subsequently sprayed on a plant surface, is efficiently taken up by a plant cell.

Cell-penetrating peptides (CPPs), also called protein transduction domains, are short peptides that facilitate the transport of cargo molecules through membranes to gain access to the cells. There are several types of cell-penetrating peptides as reviewed in Bechara and Sagan (FEBS Lett. 2013 587:1693-1702). They have the capacity to cross cellular membranes without the need of recognition by specific receptors. In general, three types can be distinguished: natural occurring peptides, fusion of different natural occurring peptides and synthetic peptides. In many cases, CPPs are coupled to cargo molecules through covalent conjugation, forming CPP-cargo complexes. To date, DNA, RNA, nanomaterials and proteins such as antibodies were reported as cargo molecules. Most studies of the complex of CPPs and protein have contributed to the applications in mammalian cells, whereas only very limited studies have focused on plant cells. It is generally known that protein introduction into plants is more difficult than protein introduction into animals. This could be because of the complicated cell wall structure of plants. The plant cell wall affects transport of big cargo molecules, and the slight negatively net charge of the cellulose could reduce interaction between the CPP and lipid bilayer by physical and the chemical manners.

The primary cell wall of land plants is composed of cellulose, hemicelluloses and pectin. Additionally, polymers such as lignin, suberin or cutin are anchored to or embedded in plant cell walls. Structural proteins (1-5%) are found in most plant cell walls; they are classified as hydroxyproline-rich glycoproteins (HRGP), arabinogalactan proteins (AGP), glycine-rich proteins (GRPs), and proline-rich proteins (PRPs). The cell wall surrounds the plasma membrane of plant cells and provides tensile strength and protection against mechanical and osmotic stress. It also allows cells to develop turgor pressure. Plant cells also have vacuoles and chloroplasts, both of which help regulate how plant cells handle water and storage of other molecules. Moreover, the biochemical composition of plant cells and plasma membranes changes during the plant growth, indicating the need to optimize various conditions to achieve delivery of cargo molecule into plant cells.

CPPs have been found to be trapped within the cell wall due to its ionic interaction with the negatively charged cell wall components (Mizuno et al. Cellular internalization of arginine-rich peptides into tobacco suspension cells: a structure-activity relationship study. J Pept Sci. 2009; 15(4):259-63. 10.1002/psc.107). Hence, CPPs for plant system must be able to penetrate the cell membrane without being absorbed onto the negatively charged cell wall. There are limited reports reporting the use of CPP for cargo (protein) delivery into plant cells, but the few reported studies were only at cellular assay level. To date, there have been limited or no reports on cargo (protein) delivery system in intact plants. The peptide-mediated delivery strategy mainly involves the formation of a peptide-fusion protein, which is achieved by the formation of carrier peptide and protein of interest by chemical cross-linking. However, these strategies tend to be labor-intensive, time-consuming, and could lead to the loss of biological activity of some of the protein cargoes. There are also studies for protein delivery in plants based on electrostatic interaction between a carrier peptide and protein with the opposite charge. Additionally, previous studies demonstrated that CPP-fused to a polycationic peptide is a more efficient nucleic acid carrier compared with that of the CPP alone in intact plants. By using the fusion peptides, the negatively charged cargo preferentially interacts with the polycationic peptide through ionic interactions, whereas the CPP interacts with fewer cargo molecules and is preferentially present on the surface of peptide-cargo complexes.

HIVTat and many other CPPs have been identified and utilized as molecular transporters in plant cells.

Below, some examples of CPP are described in more detail. While specific examples of CPPs are provided herein, the skilled person would recognize that CPPs can be selected based on criteria determined by the physicochemical properties necessary for CPPs to cross membranes. A combination of hydrophobicity, high net positive charge and the R-groups of arginine and lysine have been shown to confer membrane transduction properties to CPPs.

BP100 (KKLFKKILKYL—SEQ ID NO: 7) is an amphiphilic peptide and has CPP function. KH9 (KHKHKHKHKHKHKHKHKH —SEQ ID NO:10), R9 (RRRRRRRRR—SEQ ID NO: 8), and D-R9 (rrrrrrrrr, D-form of R9—SEQ ID NO:9) are peptides containing both CPP and cationic biomolecule binding functions. BP100(KH)9 (KKLFKKILKYLKHKHKHKHKHKHKHKHKH—SEQ ID NO: 11) and BP100CH7 (KKLFKKILKYLHHCRGHTVHSHHHCIR—SEQ ID NO: 12) are fusion peptides containing CPP and cationic sequences which are designed as stimulus-response peptides and could release the cargo molecules (peptides, protein, RNA, DNA) into the cytoplasm. KAibA(KH)9, K-alpha-aminoisobutyric acid (AibA)-AKHKHKHKHKHKHKHKHKH, and KAibA(D-R9), K-alpha-aminoisobutyric acid (AibA)-Arrrrrrrrr are synthetic peptides and containing both CPP and cationic biomolecule binding functions. KAibA (JP2019-69134 & U.S. Ser. No. 16/832,749) was chemoenzymatically synthesized using the polymerization (ACS Biomater. Sci. Eng. 2020, 6, 6, 3287-3298).

Transit peptides that have the ability to target cargo to organelles such as chloroplasts are known. Software programs have been developed to predict chloroplast subcellular localization signals. The skilled person would recognize the physicochemical properties that constitute a chloroplast transit peptide length, charge, and hydrophobicity. In the context of the present invention, a chloroplast transit sequence could be added to the nucleic acid sequence to be transported into the plant. It may also be possible to identify CPPs with transit peptides properties. Such CPPs can be used to deliver nucleic acid to plastids in either isolated plant cells or in whole plants. Also, the compositions of the present invention may comprise a CPP, a nucleic acid and an organelle targeting peptide.

The use of CPPs as effective plant macromolecular transporters shown in the present invention is important not only in monocot and dicot transgenic plant production methods, but also in plant functional genomics. The CPPs can be combined with other transduction technologies, thus making them broad based platform technologies for the manipulation of plant traits of agronomic importance. The application of CPPs to directly manipulate plant organelle genomes also facilitates organelle functional genomics.

Plants have stomata that regulate the movement of water inside the plant and out of the plant by transpiration. Each stomate is flanked by two guard cells, controlling the diameter of stoma by changing shape. Stomatal density of a leaf is under both genetic and environmental control. Stomata are generally open during the day and closed at night. Stomata are known to have a role in the uptake of solutions by a plant. Without limiting the invention, it is believed that the compositions described herein may be transported through the stomata of the plant into the plant cells. Hence, any physiological or molecular factors that influence stomata opening and closing have the potential to impact the uptake of the CPP complexes described herein. For example, the methods may be used under conditions wherein the stomata are likely to be open or in plants having large numbers of stomata. Moreover, the pH of the compositions can be adjusted to allow for more favorable uptake through the stomata. As another example, the relative humidity or temperature may be adjusted such that stomata are more likely to be open.

It is contemplated that the methods of the present invention can be used to directly incorporate nucleic acids into plant cells by spraying, it is also possible that the methods can be used in conjunction with other methods of transporting or delivering cargo into a plant cell. For example, techniques are known for the introduction of proteins for genome editing such as the CRISPR-Cas system. It may be possible to introduce proteins such as an endonuclease using such a technique while simultaneously using the compositions of the present invention to incorporate nucleic acids into plant cells.

In a first embodiment, there is provided a novel method for introducing one or more nucleic acids into a plant cell, said method comprising: (i) providing a solution of one or more nucleic acids complexed to one or more Cell Penetrating Peptides, (ii) applying the solution of (i) to a cell of a plant by spraying, and (iii) allowing the one or more nucleic acids to enter said plant cell. Preferably, the ratio of nucleic acid to Cell Penetrating Peptide ranges between 0.5 to 2.0.

The nucleic acids may include one or more of DNA, RNA, PNA and/or one or more nucleic acid analogues. The nucleic acid-CPP complex may be dissolved in water and optionally a salt and/or buffer component can be added to this solution, but this solution is preferably devoid of any further additives, such as chelators, surfactants (nonionic surfactants or ionic), humectants, or osmolytes. Therefore, in an alternative embodiment, there is provided a novel method for introducing one or more nucleic acids into a plant cell, said method comprising: (i) providing one or more nucleic acids complexed to one or more Cell Penetrating Peptides, the complex dissolved in water devoid of any further additives, (ii) applying the solution of (i) to a cell of a plant by spraying, and (iii) allowing the one or more nucleic acids to enter said plant cell. In another alternative embodiment, there is provided a novel method for introducing one or more nucleic acids into a plant cell, said method comprising: (i) providing one or more nucleic acids complexed to one or more Cell Penetrating Peptides, the complex dissolved in a salt solution, (ii) applying the solution of (i) to a cell of a plant by spraying, and (iii) allowing the one or more nucleic acids to enter said plant cell. In yet another alternative embodiment, there is provided a novel method for introducing one or more nucleic acids into a plant cell, said method comprising: (i) providing one or more nucleic acids complexed to one or more Cell Penetrating Peptides, the complex dissolved in a buffer solution, (ii) applying the solution of (i) to a cell of a plant by spraying, and (iii) allowing the one or more nucleic acids to enter said plant cell. In yet another alternative embodiment, there is provided a novel method for introducing one or more nucleic acids into a plant cell, said method comprising: (i) providing one or more nucleic acids complexed to one or more Cell Penetrating Peptides, the complex dissolved in a buffered salt solution, (ii) applying the solution of (i) to a cell of a plant by spraying, and (iii) allowing the one or more nucleic acids to enter said plant cell.

The spraying is applied directly to a plant tissue, plant organ, plant epidermis, or any other part of a plant. The plant organ may be a leaf, stem, root, or reproductive organ. The plant tissue can be any tissue, including meristematic tissue. Therefore, according to some aspects of the invention, said plant cell is part of a plant tissue, plant organ, plant epidermis, or any other part of a plant.

The Cell Penetrating Peptide can be any type of CPP. Preferably, the CPP is a cationic CPP or an amphiphilic CPP. More preferably, the cationic CPP is one of BP100 (SEQ ID NO: 7), R9 (SEQ ID NO: 8) or D-R9 (SEQ ID NO: 9), and the amphiphilic CPP is KH9 (SEQ ID NO: 10).

Thus, in another aspect, the Cell Penetrating Peptide is preferably selected from the group consisting of: BP100, KH9, R9, D-R9, BP100(KH)9, BP100CH7, KAibA(KH9) and KAibA(D-R9).

According to an aspect of the invention, the CPP is complexed with one or more of DNA, RNA, or nucleic acid analogues. Standard techniques known to the skilled person can be used for creating a nucleic acid — CPP complex. In a further aspect of the invention, the nucleic acid — CPP complex may further comprise an organelle targeting sequence. Such nucleic acid can then be delivered into an organelle such as a chloroplast or mitochondrion. The CPP may itself act as an organelle targeting peptide (see for example Cerrato et al., J. Mater. Chem. B, 2020,8, 10825-10836 or Thagun et al., Adv Sci (Weinh). 2019; 6(23): 1902064). Alternatively, the nucleic acid can be coupled to an organelle targeting peptide (MacCulloch, T. et al., Org. Biomol. Chem., 2019,17, 1668-1682).

Therefore, the present invention also provides a method of introducing a nucleic acid into a plant cell, said method comprising:
a. providing a solution of one or more nucleic acids, complexed to one or more Cell Penetrating Peptides,
b. applying said solution to a plant, plant organ or plant tissue by spraying, and
c. allowing the one or more nucleic acids to enter said plant cell;
   wherein said one or more nucleic acids are complexed to one or more Cell Penetrating Peptides and an organelle targeting peptide, or wherein the one or more nucleic acids are conjugated to an organelle targeting peptide and complexed to one or more Cell Penetrating Peptides, or wherein said one or more nucleic acids are complexed to one or more Cell Penetrating Peptides capable of targeting an organelle. In one aspect, the one or more nucleic acids are delivered into the mitochondrion, in another aspect the one or more nucleic acids are delivered into the chloroplast. Alternatively, the one or more nucleic acids can be targeted to other subcellular compartments, such as cell membranes (Jin et al. Front. Chem., https://doi.org/10.3389/fchem.2020.00824).

The methods of the present invention may be applied by spraying, nebulizing, etc. Spraying can be done on any plant part, plant organ or plant tissue. In one aspect, spraying is done on a plant leaf and in particular, spraying is done on the adaxial and/or abaxial side of a plant leaf. In one aspect, the spraying is advantageously applied to the abaxial side of a leaf. Such methods can be adapted for large scale application, for example, to crops in a greenhouse or in a field.

In another embodiment, there is provided a novel method for applying one or more nucleic acids to a plant, said method comprising: (i) providing a solution of one or more nucleic acids complexed to one or more Cell Penetrating Peptides, (ii) applying the solution of (i) to a plant by spraying, and (iii) allowing the one or more nucleic acids to enter plant cells. In yet another embodiment, there is a provided a method of applying one or more nucleic acids to a plant, said method comprising: (a) complexing said one or more nucleic acids with one or more Cell Penetrating Peptides in water devoid of any further additives; (b) applying the solution containing the one or more nucleic acids complexed with the one or more CPPs to a plant by spraying or nebulizing; and (c) allowing the complex to enter plant cells. In another alternative embodiment, there is provided a novel method for applying one or more nucleic acids to a plant, said method comprising: (i) providing one or more nucleic acids complexed to one or more Cell Penetrating Peptides, the complex dissolved in a salt solution, (ii) applying the solution of (i) to a plant by spraying, and (iii) allowing the one or more nucleic acids to enter plant cells. In yet another alternative embodiment, there is provided a novel method for applying one or more nucleic acids to a plant, said method comprising: (i) providing one or more nucleic acids complexed to one or more Cell Penetrating Peptides, the complex dissolved in a buffer solution, (ii) applying the solution of (i) to a plant by spraying, and (iii) allowing the one or more nucleic acids to enter plant cells. In yet another alternative embodiment, there is provided a novel method for applying one or more nucleic acids to a plant, said method comprising: (i) providing one or more nucleic acids complexed to one or more Cell Penetrating Peptides, the complex dissolved in a buffered salt solution, (ii) applying the solution of (i) to a plant by spraying, and (iii) allowing the one or more nucleic acids to enter plant cells.

The methods of the present invention can be applied to translocate any nucleic acid.

In one particular aspect, the nucleic acid or nucleic acid analogue is capable of modulating gene expression. Therefore, in another aspect, there is provided a method of modulating gene expression in a plant cell, said method comprising providing a solution according to the present invention, comprising one or more nucleic acids capable of modulating expression of a gene, complexed to one or more CPPs; applying said solution to a plant, plant organ or plant tissue by spraying; and allowing the one or more nucleic acids capable of modulating expression of a gene to enter said plant cell and modulate gene expression. The present invention thus provides a method of modulating gene expression in a plant cell, said method comprising:
a. providing a solution comprising one or more nucleic acids capable of modulating expression of a gene, complexed to one or more Cell Penetrating Peptides;
b. applying the solution to a plant, plant organ or plant tissue by spraying; and
c. allowing the one or more nucleic acids capable of modulating expression of a gene to enter said plant cell and to modulate gene expression. Alternatively, the nucleic acids capable of modulating expression of a gene, complexed to one or more Cell Penetrating Peptides are dissolved in water, or in water supplemented with salt and/or buffer.

Advantageously, the methods of the invention can be carried out using spraying techniques such as dripping, nebulizing, atomizing, misting or any other form of application wherein the solution is applied without directly contacting the plant.

Advantageously, the methods of the invention can be carried out under conditions that are favorable to stomatal opening, or under conditions where stomata are open prior to the application of the solution.

Advantageously, the methods of the invention may encompass multiple applications, wherein the solution is allowed to enter the plant cells for a period of time, followed by consecutive application of additional solution by way of spraying.

Advantageously, the methods of the invention comprises application to a field crop for selectively controlling the growth of weeds wherein the solution of one or more nucleic acids complexed to one or more Cell Penetrating Peptides further comprises a herbicide or other pesticide.

The present invention also encompasses use of a solution comprising a Cell Penetrating Peptide complexed with one or more nucleic acids for the control of a physiological process in plants, including but not limited to flower induction, resistance to pests and sensitivity to growth regulators, wherein the solution is applied to the plant by spraying.

Advantageously, the methods of the invention can be applied for genome editing of plants using a CRISPR-Cas system, wherein the nucleic acid comprises a guide RNA.

Advantageously, the methods of the invention can be applied to a plant concurrently with another transfection method.

Unless defined otherwise, technical and scientific terms as used herein have the same meaning as commonly understood by one of ordinary skill in the art. One skilled in the art will recognize many methods can be used in the practice of the present disclosure. Indeed, the present disclosure is in no way limited to the methods and materials described. Moreover, the present disclosure is not intended to be limited by any particular scientific theory. For purposes of the present disclosure, the following terms are defined below.

As used herein “spraying” refers in general to any technique that may be used to achieve roughly uniform distribution of a liquid compound or a dissolved compound, and/or that may be used to spread liquid in small drops or thin jets over an area. Spraying also includes application by nebulizing or by mist or aerosol. When used in connection with a crop application, “spraying” includes crop dusting (aerial), and may make use of for example hydraulic sprayers, mist blower, electrostatic sprayer, rotary disk sprayer, spray gun, trolley, thermal fogger, mechanical fogger https://ag.umass.edu/greenhouse-floriculture/fact-sheets/sprayers-spray-application-techniques) hand-operated or motorized, blower sprayer (e.g. the sprayer mainly consists of high-pressure piston pump, which atomise the spray solution, axial or centrifugal fan to produce a stream of air). Any technique used in aeroponics or fogponics can advantageously be used. When spraying leaves, the spraying or misting may be applied to the underside of a plant surface (e.g. the abaxial surface of a leaf) or to the adaxial surface of a leaf or to both sides simultaneously. The speed of application can be adjusted as appropriate to ensure appropriate application, as can the angle of spray. Precision spraying techniques may also be used (i.e. automated target detecting systems). Such techniques are particularly useful when the methods of the present invention are employed as a means of weed control. Spraying or other means of applying can be carried out in a semi-automated or automated such as by a drone or any type of robot.

“TAMRA” refers to 5-Carboxytetramethylrhodamin, a widely used fluorophore for preparing bioconjugates and in the context of the present invention, TAMRA is conjugated with CPPs. Alternative fluorophores useful for conjugation are known to the skilled person.

The term “nucleic acids” and “nucleotides” refer to naturally occurring or synthetic or artificial nucleic acid or nucleotides, including single- or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. The terms “nucleic acids” and “nucleotides” comprise deoxyribonucleotides or ribonucleotides or any nucleotide analogue and polymers or hybrids thereof in either single- or double-stranded, sense or antisense form. In case the nucleic acid is DNA; the term “nucleic acid” can be non-coding or coding (such as genes or cDNA), can be short (oligonucleotides) or long, can be linear or circular, can be in B-DNA, A-DNA or Z-DNA conformation. In case the nucleic acid is RNA, the term covers any type of RNA, including but not limited to mRNA, tRNA, rRNA, dsRNA, ribozymes, circular RNA, and various types of regulatory RNAs like miRNA, IncRNA, enhancer RNA, RNA associated with the CRISPR-Cas system (crRNA, tracrRNA or sgRNA).

The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Nucleic acid analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

The methods of the invention are advantageously applicable to any plant, in particular to any plant as defined herein. Plants that are particularly useful in the methods of the invention include all plants which belong to the superfamily Viridiplantae, in particular monocotyledonous and dicotyledonous plants including fodder or forage legumes, ornamental plants, food crops, trees or shrubs. According to one embodiment of the present invention, the plant is a crop plant. Examples of crop plants include but are not limited to chicory, carrot, cassava, trefoil, soybean, beet, sugar beet, sunflower, canola, alfalfa, rapeseed, linseed, cotton, tomato, potato, Stevia species such as but not limited to Stevia rebaudiana and tobacco. According to another embodiment of the present invention, the plant is a monocotyledonous plant. Examples of monocotyledonous plants include sugarcane. According to another embodiment of the present invention, the plant is a cereal. Examples of cereals include rice, maize, wheat, barley, millet, rye, triticale, sorghum, emmer, spelt, einkorn, teff, milo and oats. In a particular embodiment the plants of the invention or used in the methods of the invention are selected from the group consisting of maize, wheat, rice, soybean, cotton, oilseed rape including canola, sugarcane, sugar beet and alfalfa.

The invention also extends to harvestable parts of a plant such as, but not limited to seeds, leaves, fruits, flowers, stems, setts, sugarcane gems, roots, rhizomes, tubers and bulbs, which harvestable parts which have been treated using a method according to the present invention, which harvestable parts have modified, preferably improved, characteristics compared to harvestable parts which have not been treated. In particular, such harvestable parts are roots such as taproots, rhizomes, fruits, stems, beets, tubers, bulbs, leaves, flowers and/or seeds.

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, callus tissue, embryos, meristematic regions, gametophytes, sporophytes, pollen and microspores, or propagules.

“Propagation material” or “propagule” is any kind of organ, tissue, or cell of a plant capable of developing into a complete plant. “Propagation material” can be based on vegetative reproduction (also known as vegetative propagation, vegetative multiplication, or vegetative cloning) or sexual reproduction. Propagation material can therefore be seeds or parts of the nonreproductive organs, like stem or leave.

### BRIEF DESCRIPTION OF THE DRAWINGS

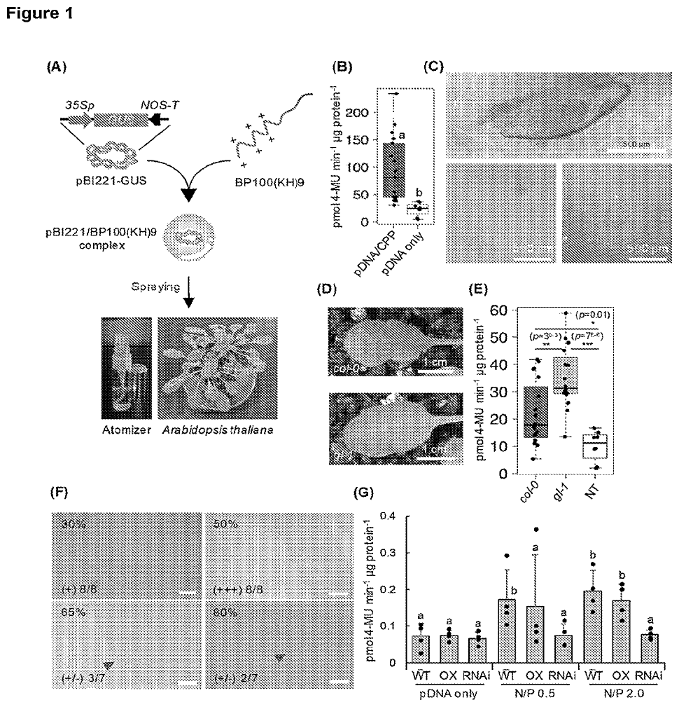

FIG. 1. Uptake Efficiency of Cell-Penetrating Peptide-Based DNA Nanocarriers to Plant Leaf After Spray Application
(A) Sprayable peptide-based DNA delivery to Arabidopsis thaliana mediated by cell-penetrating peptide (CPP; BP100(KH)9). The BP100(KH)9/pB1221 complexes were formed in aqueous solution and applied on plant leaves using spray atomizer. The activity of GUS reporter was assayed after 24 hours post spraying. (B) GUS activity in plant leaves transfected with CPP/pDNA complexes after spraying for 24 hours. The distribution of GUS activity in at least 20 sprayed leaves are shown as a box plot. Black bars represent median of GUS activity. Dots represent each data point. Letters indicate the significant differences of GUS activity analyzed by one-way ANOVA with Tukey's HSD test at p=0.05. (C) Histochemical staining of GUS reporter in plant leaves sprayed with BP100(KH)9/pB1221 complex solution. (D) Abundance of trichrome on col-0 and g1-1 Arabidopsis mutant. (E) GUS activity in plant leaves transfected with BP100(KH)9/pB1221 complex via spraying. Asterisks represent the levels of significant difference of GUS activity in two samples analyzed by Student's t-test. NT is non-transfected col-0 leaves. (F) GUS staining in plant leaves after spraying with BP100(KH)9/pB1221 complexes and kept at different humidity conditions. Plus/minus shows the GUS signal in plant leaves observed by stereomicroscopy. Proportion of number represents number of GUS-positive samples per total number of leaves collected for the analysis. Red arrows indicated the GUS-positive spots in plant leaves. Scale bars=500 μm. (G) GUS activity in transgenic Arabidopsis leaves sprayed with BP100(KH)9/pB1221 complex solutions. WT=wild type, OX=STOMAGEN-overexpressor, and RNAi=STOMAGEN-suppressor. Dots represent the distribution of each data point. Error bars=standard deviation (SD). Letters indicate significant differences of mean analyzed by one-way ANOVA with Tukey's HSD test at p=0.05.

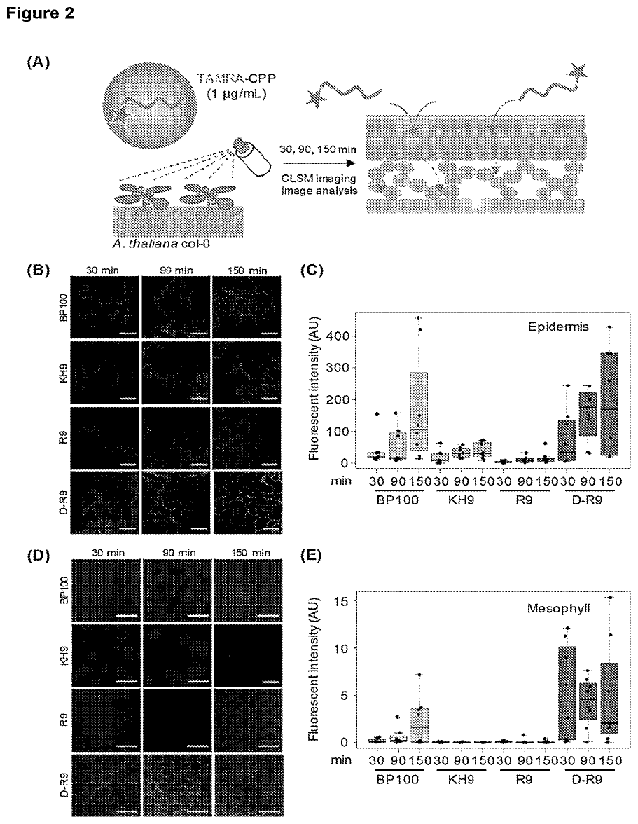

FIG. 2. Translocation of TAMRA-CPPs to Arabidopsis Leaf Cells
(A) Spray application of TAM RA-CPP solution on fully expanded Arabidopsis leaves. After periods of incubation, TAM RA-fluorescent signal was determined in epidermal cells and palisade mesophyll cells on the adaxial side of leaf under confocal fluorescence microscope. (B) Fluorescent microscopic observation of TAMRA-CPPs in Arabidopsis leaf epidermal cells after spraying. Scale bars=50 μm. (C) Fluorescent intensity of TAMRA-CPPs in epidermal cells after spraying. The distributions of fluorescent intensities in epidermal cells of Arabidopsis leaves in 8 areas of interest (ROI) were shown as box plot. Black bars represent medians of intensity values. (D) Microscopic autographs of various TAM RA-CPPs in Arabidopsis palisade mesophyll cell layer. Scale bars=50 μm. (E) Box plot represents the distribution of fluorescent intensities in mesophyll cells of 8 ROIs.

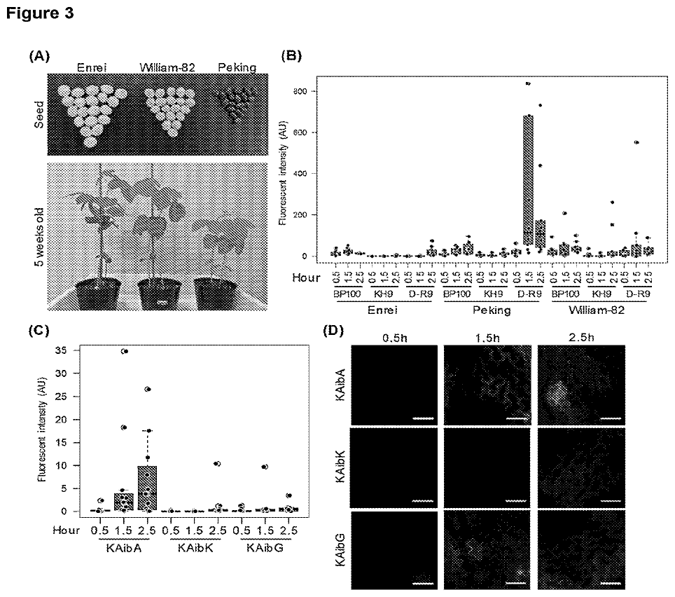

FIG. 3. Translocation of TAMRA-CPPs to Soybean Leaf
(A) Plant physiologies of three different commercially available soybean cultivars. Seeds and 5-week old soybean plants of cultivars Enrei, Peking, and William-82 were germinated and cultured in the same cultivation conditions. (B) Distributions of fluorescent intensities in soybean leaves after spraying with TAM RA-CPPs solution and washing. The distribution of fluorescent intensity data was shown as box plot with median (black bar). Dots represent each data point in the distribution (n=9). (C) Fluorescent intensities of chemoenzymatically-synthesized TAMRA-alpha-aminoisobutyric acid (Aib)-containing CPPs in soybean (cultivar Enrei) leaves after spraying (n=12). The data was shown as box plot. (D) Fluorescent images of TAMRA-alpha-aminoisobutyric acid (Aib)-containing CPPs in soybean leaves. Scale bars=50 μm.

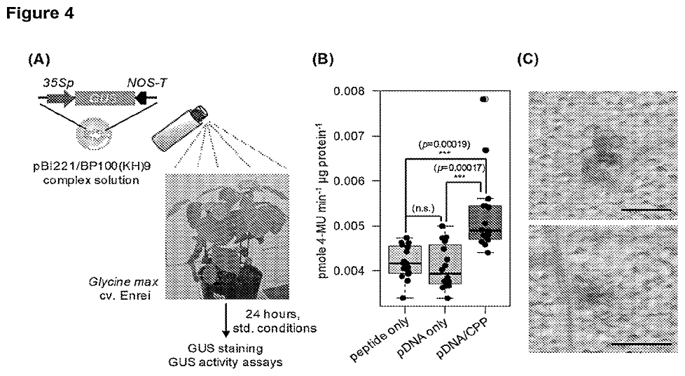

FIG. 4. Transfection Efficiency of Spray-Applied pDNA/CPP Complex in Soybean Leaf
(A) Foliar application of pB1221/BP100(KH)9 complex solution to soybean leaves (cultivar Enrei). (B) GUS activity in soybean leaves sprayed with BP100(KH)9/pB1221 complex solution at 24 hours post spraying. The distribution of data was illustrated by box plot. Black bars represent the median of distributed data. Dot indicates each data points in the analysis (n=16). Different number of asterisks shows differences in levels of statistical significance. n.s. =no statistically significant difference. (C) Expression of GUS reporter protein in soybean leaf cells stained with GUS staining solution. Scale bars=500 μm.

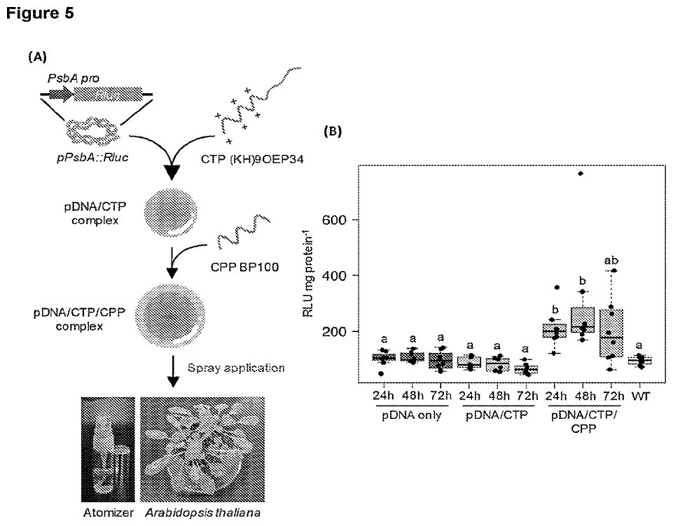

FIG. 5. Targeted DNA Delivery to Arabidopsis Chloroplast Using Peptide Carrier-Based Spray Application
(A) Formation of clustered pDNA/chloroplast-targeting/cell-penetrating peptide (pDNA/CTP/CPP) nanocarrier. The complex of pPsbA::Rluc (chloroplast-specific expression vector) with CTP (KH)90EP34 were formed in Milli-q water for specific targeting of pDNA delivery to chloroplasts. CPP BP100 was subsequently added to pDNA/CTP complex solution to enhance cell penetration efficiency of the resulting pDNA/CTP/CPP complex to plant cell. (B) After spraying with clustered pDNA/CTP/CPP nanocarriers and control solutions containing pDNA only and pDNA/CTP complex, Arabidopsis leaves were collected and Renilla luciferase (Rluc) activity was assayed at 24, 48, and 72 hours post spraying. The distribution of Rluc activities in plant leaves are shown as box plot with median (black bar). Dots in the plot represent each data point (n=8). WT=wild type (non-sprayed leaves). Letters indicate significant differences of mean of Rluc activity among the treatments (one-way ANOVA with Tukey's HSD test at p=0.05).

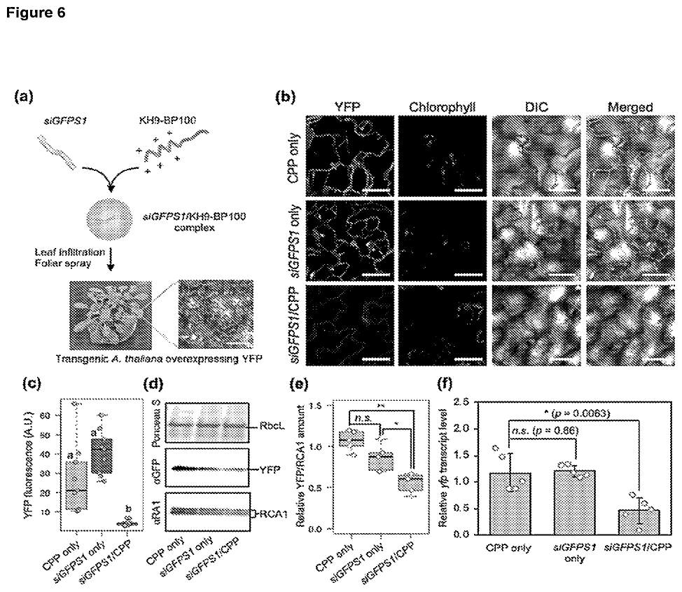

FIG. 6. Suppression of Gene Expression in Plant Cell Mediated by Sprayable Peptide-Based siRNA Cargos
(a) Formulation of siGFPS1/KH9-BP100 complex for yfp gene suppression in transgenic Arabidopsis plants. (b) YFP fluorescence in plant cells at 3-days post spraying with solution containing siGFPS1/KH9-BP100 complex. Scale bars=50 μm. (c) Quantitative fluorescent intensity of YFP in plant cells at 3-days post spraying with siRNA/CPP complex. The distributions of YFP fluorescence from 9 regions of interest (n =9) were shown as box plot. Dots represent the fluorescent values. Black bars are median of the distributed data. Letters indicate significant differences of mean analyzed by one-way ANOVA with Tukey's HSD test at p=0.05. (d) Immunoblot analysis of YFP and endogenous RubisCo Activase 1 protein (e.g. RCA1; a highly abundance plant intracellular protein) in soluble proteins extracted from Arabidopsis leaves after 3 days of spraying with siRNA/CPP complex. The membrane was stained with Ponceau S staining solution prior probing with antibodies. RbcL=RubisCo large subunit. (e) Relative YFP abundance in total leaf protein after 3 days of spraying with siRNA/CPP complex determined by immunoblotting. Relative amounts of YFP to RCA1 were shown as box plot. Circles represent the distribution of data points in box plot. Black bars are median of distributed values. (f) Relative yfp transcript levels in plant leaf at day 3 post spraying with siRNA/CPP complex. Error bars =standard deviation. Circles represent variations of relative yfp transcript levels in 5 different experiments. Statistical differences in (e) and (f) were analyzed by comparative Student's t-test (n=5). *, **=statistically significant difference at p≤0.01 and p 0.001, respectively. n.s.=no significant difference.

### EXAMPLES

### Chemicals and Common Methods

Unless indicated otherwise, cloning procedures carried out for the purposes of the present invention including restriction digest, agarose gel electrophoresis, purification of nucleic acids, ligation of nucleic acids, transformation, selection and cultivation of bacterial cells were performed as described (1). Sequence analyses of recombinant DNA were performed with a laser fluorescence DNA sequencer (Applied Biosystems, Foster City, CA, USA) using the Sanger technology (2). Unless described otherwise, chemicals and reagents were obtained from Sigma Aldrich (Sigma Aldrich, St. Louis, USA), from Promega (Madison, WI, USA) or Invitrogen (Carlsbad, CA, USA). Restriction endonucleases were from New England Biolabs (Ipswich, MA, USA) or Roche Diagnostics GmbH (Penzberg, Germany). Oligonucleotides are synthesized by IDT (Coralville, IA, USA).

### Identification of Sequences Related to the Nucleic Acid Sequence Used in the Methods of the Invention

Sequences (full length cDNA, ESTs or genomic) related to the nucleic acid sequence used in the methods of the present invention were identified amongst those maintained in the Entrez Nucleotides database at the National Center for Biotechnology Information (NCBI) and other databases using database sequence search tools, such as the Basic Local Alignment Tool (BLAST) (3, 4). The program is used to find regions of local similarity between sequences by comparing nucleic acid or polypeptide sequences to sequence databases and by calculating the statistical significance of matches. For example, the polypeptide encoded by the nucleic acid used in the present invention was used for the TBLASTN algorithm, with default settings and the filter to ignore low complexity sequences set off. The output of the analysis was viewed by pairwise comparison, and ranked according to the probability score (E-value), where the score reflects the probability that a particular alignment occurs by chance (the lower the E-value, the more significant the hit). In addition to E-values, comparisons were also scored by percentage identity.

Percentage identity refers to the number of identical nucleotides (or amino acids) between the two compared nucleic acid (or polypeptide) sequences over a particular length. In some instances, the default parameters may be adjusted to modify the stringency of the search. For example, the E-value may be increased to show less stringent matches. This way, short nearly exact matches may be identified.

Escherichia coli was used as propagation microorganism for all the plasmids used in our experiments, as well as for further propagation, maintenance of the modified targets, and heteroexpression of target protein. E. coli was grown according standard microbiological practices (5).

### Example 1

### Generation of Labelled-Cell Penetrating Peptides (CPP) Solution and CPP-Nucleic Acid Solution

Cell-penetrating peptides (CPPs), also called protein transduction domains, are short peptides that facilitate the transport of cargo molecules through membranes to gain access to the cells. In many cases, CPPs are coupled to cargo molecules through covalent conjugation, forming CPP-cargo complexes. To date, DNA, RNA, nanomaterials and proteins such as antibodies were reported as cargo molecules. Most studies of the complex of CPPs and protein have contributed to the applications in mammalian cells, whereas only very limited studies have focused on plant cells. This could be due to the complicated cell wall structure of plant shows intransigence on internalization such big cargo molecules, and the slight negatively net charge of the cellulose could reduce interaction between the CPP and lipid bilayer by the physical and the chemical manners. The plant cells are mainly containing cellulose, hemicellulose and pectin. These biochemical compositions are changing during the plant growth, indicating that optimization of various conditions to achieve delivery of cargo molecule into plant cells is needed. BP100 (KKLFKKILKYL—SEQ ID NO: 7) is an amphiphilic peptide and has CPP function. KH9 (KHKHKHKHKHKHKHKHKH—SEQ ID NO: 10), R9 (RRRRRRRRR SEQ ID NO: 8), and D-R9 (rrrrrrrrr, D-form of R9 - SEQ ID NO: 9) are peptides containing both CPP and cationic biomolecule binding functions. BP100(KH)9 (KKLFKKILKYLKHKHKHKHKHKHKHKHKH—SEQ ID NO: 11) and BP100CH7 (KKLFKKILKYLHHCRGHTVHSHHHCIR—SEQ ID NO: 12) are fusion peptides containing CPP and cationic sequences which are designed as stimulus-response peptides and could release the cargo molecules (peptides, protein, RNA, DNA) into the cytoplasm. KAibA(KH)9, K-alpha-aminoisobutyric acid (AibA)-AKHKHKHKHKHKHKHKHKH, and KAibA(D-R9), K-alpha-aminoisobutyric acid (AibA)-Arrrrrrrrr are synthetic peptides and containing both CPP and cationic biomolecule binding functions. KAibA (U.S. Ser. No. 16/832,749) was chemoenzymatically synthesized using the polymerization reaction (ACS Biomater. Sci. Eng. 2020, 6, 6, 3287-3298). TAMRA-CPPs are CPPs that labeled by fluorescent molecule 5-carboxytetramethylrhodamine (TAMRA) at the C-terminal end and were used for optimization of various conditions. For the labelling of CPP with TAMRA, the amino acids in Boc-CPPs were deprotected using tri-fluoroacetic acid (TFA). A solution of tetramethylrhodamine-5-isothiocyanate in dimethyl sulfoxide (DMSO) was added to CPP solutions and kept stirring at 25° C. for 14 h. The solution was centrifuged at maximum speed at 4° C. for 60 minutes and the precipitate was washed with Milli-Q water. After lyophilizing, a pink solid was obtained. Dried precipitate of TAM RA-CPP was re-suspended with Milli-q and the TAMRA-CPP solution was kept at 1.0 mg/mL concentration in −80° C. Plasmid DNA that contains expression cassette of reporter gene such as GUS (SEQ ID NO: 1), GFP (SEQ ID NO: 2), DsRed (SEQ ID NO: 3), and Renilla luciferase (Rluc—SEQ ID NO: 4) was used to detect successful delivery into plant cells. Plasmid DNA that contains expression cassette of herbicide tolerant gene such as PPO (SEQ ID NO: 6)or ALS (SEQ ID NO: 5) was used for further application. Gene expression cassettes containing either CaMV 355 promoter or chloroplast specific promoter; PsbA or rrn16S gene promoter, gene of interest, and NOS terminator, were cloned into pB1221 vector. Plasmid DNA with concentration at 1.0 mg/mL was prepared by Maxi prep (QIAGEN) according to the manufacture protocol. To prepare the CPP-DNA solution, plasmid DNA (1.0 mg/mL) was mixed with CPP (1.0 mg/mL) at various N/P ratios. For BP100(KH)9-DNA complex, the complex was prepared in N/P ratio at 0.5, 1.0, 1.5 and 2.0. The complex solutions were pipetted gently and incubated at RT for 30 min in the dark. This solution was adjusted to final volume of 5 mL by adding autoclaved Milli-q water, and then continuing incubation under the same condition for another 30 min. Each solution was repeatedly pipetted and used for spraying application.

### Example 2

### Plant Growth Conditions

Seeds of Arabidopsis thaliana ecotype col-0, mutant lines gl-1 and over expressor and transcriptional repressor of STOMAGEN gene, STOMAGEN-Ox and STOMAGEN-RNAi, were germinated in pots with planting medium containing a mixture of soil (Pro-Mix; Premier Tech Ltd, Quebec, Canada) and vermiculite in a ratio of 2:1. Plants were grown and incubated under 16 hours light/8 hours dark at 21° C. in a plant incubator (Biotron NK System, Osaka, Japan). Two weeks old plants were used for experiments.

Seeds of soybean (Glycine max) cultivars Enrei, William-82, and Peking, were germinated between germination papers that has been moistened with tap water. Then the seeds with papers were placed in a plastic bag and incubated in a growth chamber under 18 hours light/6 hours dark at 26° C. for three days. One and a quarter tea spoon of Multicoat 4 was mixed with 2 L of autoclaved sand, which was then moistened with tap water. Three days old soybean seedlings were then transplanted into containers filled with moist sand mix and incubated in the same growth chamber. Leaves from 5 weeks old plants were used for experiments.

Seeds of tomato (Solanum lycopersicum) were germinated on wet filter paper, and then moved to pots with planting medium containing a mixture of soil. Plants were grown and incubated under for 16 hours light at 28° C/8 hours dark at 20° C. in a plant incubator. Leaves from 2-month-old plants were used for experiments.

### Example 3

### Test of Various Spray Conditions

For the initial testing and optimizing the spray condition, one milliliter of BP100(KH)9/pB1221-GUS complex solution was sprayed on leaves of Arabidopsis thaliana ecotype col-0 (FIG. 1A). Arabidopsis plants sprayed with BP100(KH)9/pB1221-GUS complex were kept at standard culture conditions for 24 hours. Leaves of Arabidopsis sprayed with BP100(KH)9/pB1221-GUS complexes showed significantly higher GUS activity than that in the leaves sprayed with the solution containing pB1221-GUS plasmid DNA (pDNA) only (FIG. 1B and C). To access whether leaf trichrome is the target cell of the CPP-based nanocarrier transfection of plasmid DNA, a solution containing BP100(KH)9/pB1221-GUS complexes was sprayed on leaves of the non-glandular trichrome Arabidopsis mutant, gl-1 (FIG. 1D). Spray application of BP100(KH)9/pB1221-GUS complex showed enhanced GUS activities in both col-0 and gl-1 Arabidopsis leaves (FIG. 1E). Stomatal number and aperture play significant roles in biomolecule translocation to plant leaf tissue and cell layer. The feasibility of BP100(KH)9/pB1221-GUS nanocarrier uptake in different stomata opening stages were determined in various humidity conditions by spraying complex solution on Arabidopsis col-0 leaves. Spray-treated plants were then cultured in humidity chambers with different percentage of relative humidity (% RH) for 24 hours. After incubation in the humidity chamber, plant leaves were stained with GUS histochemical staining solution. Leaves incubated at 50% RH had maximum GUS signal with the highest detection frequency (FIG. 1F). However, changing the humidity conditions decreased both GUS intensity and detection frequency (FIG. 1F). To further study the role of leaf stomata on nanocarrier uptake, the BP100(KH)9/pB1221-GUS complex solutions were sprayed on leaves of transgenic Arabidopsis thaliana which manifest different number of stomata per area (overexpressor and transcriptional repressor of STOMAGEN gene; STOMAGEN-Ox and STOMAGEN-RNAi, respectively). No enhanced GUS activity was observed in plant leaves treated with solutions containing pDNA only (FIG. 1G). GUS activities were significantly increased in leaves of wild type and STOMAGEN-Ox transgenic line after spraying with BP100(KH)9/pB1221-GUS complexes formed in N/P ratios=0.5 and 2.0 (FIG. 1G). These results suggest that uptake of peptide/biomolecule cargo is a stomatal-dependent phenomenon.

In summary, we successfully transformed CPP/pDNA complex into Arabidosis leaf by spraying and observed GUS protein expression. Existing trichome or not in the leaf surface didn't make any difference in terms of delivery efficiency. Humidity of 50% was the most efficient condition, and decreasing or increasing the humidity decreased the delivery efficiency. Existing stomata in the leaf surface was critical to have the efficient delivery.

### Example 4

### Examining Penetrating Efficiency on Different Type of CPPs

To identify the CPP that shows highest penetrating efficiency using spray method in Arabidopsis leaf, TAMRA-BP100, TAMRA-KH9, TAMRA-R9, and TAMRA-D-R9 (these CPPs were labelled with TAMRA fluorophore) were used. Each TAMRA-CPP was adjusted as concentration at 1 mg/L in water. One millilitre of TAMRA-CPP solution was applied at a leaf surface. Leaf surface was washed three times with water after 30, 90 and 150 min post spraying, and the intensity of fluorescence was measured by CLSM imaging analysis (FIG. 2A). The fluorescent intensity increased over the time in epidermal cells (FIG. 2B and C). BP100 and D-R9 showed higher cell penetrating efficiency compared with KH9 and R9 (FIG. 2B and C). The fluorescent intensities of TAMRA-BP100 and TAMRA-D-R9 at 150 min showed 5-fold higher than the intensity at 30 min (FIG. 2B and C). To identify the depth of penetration, the intensity of fluorescence in mesophyll cell layer. On mesophyll cells, D-R9 showed the highest penetrating efficiency (FIG. 2D and E).

In summary, the cell penetrating efficiency was gradually increased over the incubation time in Arabidopsis leaf. We observed significant difference of cell penetrating efficiency between CPPs.

BP100 and D-form of R9 (D-R9) showed higher efficiency compared with KH9 and R9. In addition to that, D-R9 was able to penetrate deep inside of the tissue and stay longer without degradation.

### Example 5

### Introduction of DNA Coding PPO Gene into Soybean (Glycine max) Leaf by Spraying CPP and Plasmid DNA Solution

Soybean leaves are different in both architecture and leaf chemical components. We further examined the translocation efficiency of highly efficient native CPP sequences in soybean leaves. Leaves from 5 weeks old plants were used for the experiment. Solutions containing 1.0 mg/mL of natural TAMRA-CPP; TAMRA-BP100, TAMRA-KH9, TAMRA-R9, and TAMRA-D-R9 were sprayed on soybean leaves of 3 different cultivars (Enrei, Peking, and William-82) and incubated for 30 min, 90 min, and 150 min (FIG. 3A). The fluorescence imaging and image analysis were conducted to determine the translocation efficiency of these CPPs to plant leaves. The fluorescence intensity of TAMRA-CPPs was gradually increased over the incubation time (FIG. 3B). Different soybean cultivars responded differently to the translocation of CPPs to plant cells. Among the three cultivars, Peking showed the highest susceptibility to the translocation of all three CPPs to plant cells (FIG. 3B). Considering the translocation efficiencies of these three CPPs, TAMRA-D-R9 showed the highest fluorescence intensity in epidermal cells of leaves of all three soybean cultivars (FIG. 3B). This result suggested that there are significant efficiency differences between conditions, and there is a room to improve.

The chemoenzymatically-synthesized poly-alpha-aminoisobytyric acid (Aib)-contained CPPs showed remarkable activity to translocate across the tough plant cell boundaries. We tried to determine the efficiency of artificial CPPs containing Aib to soybean leaves using spraying. KAibA, KAibK, and KAibG were synthesized and labelled with TAMRA as described in Example 1. The solutions containing 1 mg/mL of these artificial TAMRA-CPPs were sprayed to fully expanded leaves of 5-weeks old soybean cultivar Enrei and the fluorescence imaging was carried out at 30 min, 90 min, and 150 min after spraying. FIG. 3C shows the fluorescent intensity of artificial TAMRA-CPPs in soybean leaves. TAMRA-KAibA showed the strongest fluorescence compared to KAibK and KAibG (FIG. 3C and D). These fluorescent intensities in TAMRA-KAibA-sprayed leaf epidermal cells were progressively increased as increasing the incubation time. However, the average fluorescent intensity in TAMRA-KAibA-treated soybean leaves was lower than that previously achieved by TAMRA-D-R9 (FIG. 3B and C).

Transfection of pB1221-GUS to soybean leaves mediated by CPP/pDNA cargos was carried out using the same protocol as in Arabidopsis (FIG. 4A). GUS histochemical staining and enzymatic assay in transfected leaves were performed after 24 hours post spraying. GUS activity assay result suggested that CPP/pDNA complex-based transfection protocol developed for Arabidopsis is able to transform soybean leaf cells (FIG. 4B). GUS activity in soybean leaves sprayed with BP100(KH)9/pB1221-GUS complex was significantly higher than that in the leaves sprayed with peptide and pDNA only (FIG. 4B). GUS staining indicated that expression of GUS is epidermal cell-specific (FIG. 4C), suggesting lower penetration ability of this CPP/pDNA complex in soybean leaf cell than in the Arabidopsis cells. Using this optimized condition, PPO gene was delivered into 5 weeks old soybean leaves of cultivar Peking. Plasmid DNA which contains PPO expression cassette was mixed with D-R9 and incubated for 30 min. The solution was sprayed on the leaf surface. Twenty-four hours after the spray, the leaf surface was washed with water three times. Then leaf tissue was immediately frozen. RT-PCR and western blot analysis were performed to examine the PPO gene expression and PPO protein accumulation. As a control, the empty vector was sprayed at the same time. Three biological replications, three independent experiments were done.

In summary, we successfully delivered pDNA into soybean leaf by CPP-pDNA complex spraying method. However, the efficiency was lower than that of Arabidopsis leaf in conditions that we tested here. The cell penetrating efficiency gradually increased over the incubation time same as that of Arabidopsis leaves. We observed BP100 and D-R9 showed higher cell penetrating efficiency in general, and the cultivar Peking and D-R9 combination showed the most efficient cell penetrating effect.

### Example 6

### Targeted Gene Delivery into Arabidopsis Chloroplast by Spraying Solution Containing Chloroplast-Targeting Peptide, and CPP BP100

Next, we attempted to deliver DNA to a specific organelle in the cell using CPP spray method. We chose chloroplast as a target organelle. Approximately 100 chloroplasts exist per epidermal cell in Arabidopsis leaf. Chloroplast-targeting peptide, (KH)90EP34; KHKHKHKHKHKHKHKHKHMFAFQYLLVM was used for chloroplast-specific gene delivery into Arabidopsis leaf. Plasmid DNA (pDNA) which contains a Renilla luciferase (Rluc) gene expression cassette under transcriptional regulation with PsbA gene promoter and chloroplast-targeting peptide (CTP), (KH)90EP34, were first mixed together and incubated for 30 min to form pDNA/CTP complex in N/P ratio=1.0 (FIG. 5A). Then cell-penetrating peptide (CPP), BP100, was added and further incubated for 30 min to form the clustered pDNA/CTP/CPP complex in N/P ratio=1.0 (FIG. 5A). The clustered pDNA/CTP/CPP complex solution was sprayed on fully expanded Arabidopsis leaf surface using spray atomizer (FIG. 5A). At 24, 48, and 72 hours after spraying, the leaf was washed three times with water, and Rluc activity in sprayed plant leaves was assayed. Rluc activity in plant leaves sprayed with pDNA only and pDNA/CTP complex was not significantly different from the basal level in non-transfected leaves (FIG. 5B). When we deliver pPsbA::Rluc using clustered CTP/CPP carrier, Rluc activity was 2-10-fold higher than the values in control experiments. (FIG. 5B). Interestingly, this expression of Rluc gene in clustered pDNA/CTP/CPP complex-sprayed plant leaves were maintained up to 72 hours post spraying (FIG. 5B).

With these results, we concluded that the sprayable peptide-based biomolecule application technique can apply for organelle targeted application as well.

### Example 7

### RNA Delivery and Local Gene Silencing in Arabidopsis Leaf

Transient RNA interference (RNAi) technology is an outstanding tool for plant biotechnology to emphatically engineer the function of a target plant metabolic process. Artificial small RNA molecules such as short-interference RNA (siRNA), micro-RNA, (miRNA), and short-hairpin RNA (shRNA) have abilities to transcriptionally-suppress the expression of plant metabolic enzymes (6). However, these short RNA molecules are sensitive to the histrionic RNA degradation process in plant cells. CPPs demonstrate their function in fortifying the interacting RNA molecules against RNA degradation process. Free siRNA molecules applied to plant cells using high-pressure spraying consequentially suppressed the expression of target mRNA molecules (7). Additionally, recent studies demonstrated that conjugating small RNA molecules with NPs such as CPP (8), 3-dimentional (3-D) DNA nanostructures (9), and carbon nanotubes (10) enhanced gene silencing efficiency and stability of RNA molecules in plant cells after infiltration. Hence, we attempted to develop a high-throughput spray application technique to apply siRNA/peptide complex to plant cells for efficient gene knockdown. We synthesized 27-bp siGFPS1 RNA duplexes which showed superior activity to silence GFP synthesis in the transfected cells (11). To test gene silencing function of siGFPS1, the synthetic double-stranded siRNA molecules were formed complexes with KH9-BP100 and syringe-infiltrated to transgenic Arabidopsis leaves overexpressing yellow-fluorescent protein (YFP) (FIG. 6a). At day-3 post infiltration, we observed significant reductions of YFP fluorescence and protein accumulation in leaves infiltrated with siGFPS1/KH9-BP100 complexes.

Intriguingly, spraying of siGFPS1/KH9-BP100 complex solution to YFP overexpression plants drastically reduced YFP fluorescence in plant cells after 3 days post application (FIG. 6b and c). We examined 45.5% decrease of YFP protein and 54.1% reduction of yfp transcripts in leaves sprayed with siRNA/CPP complexes at day-3 of spraying (FIG. 6d to f) which is comparable to the efficiency previously achieved by double-stranded GFP5/CPP complex infiltration (8). However, this efficiency is lower than that of 3-D DNA nanostructures-, carbon dots- and carbon nanotubes-mediated GFP silencing in plant cells infiltrated or sprayed by siRNA/NP conjugates (7,9,12). This could be due to the different physicochemical properties, surface coating chemistries, and cellular uptake and distribution of different NPs. Taken together, our results suggest a potential use of CPP-mediated siRNA spraying for suppression of target protein in plant cells. This peptide carrier-based RNAi foliar spraying enables a high-throughput application in plant metabolic engineering, non-transgenically.

In summary, we concluded that RNAs can be delivered with avoiding the degradation into Arabidopsis leaf with CPP spray method and were able to play a role in the local gene expression suppression.

### Example 8

### Large Scale and Field Application for Agriculture Use

The spray method is applicable in a large-scale field application. Delivering and expressing herbicide tolerant gene such as ALS, and PPO or insect resistant genes on leaf surface in economically important crops is a great example. First the solution containing biologically active molecules such as plasmid DNA harbouring a gene expression cassette, RNA molecules and proteins and BP100(KH)9 is sprayed on soybean field using controlled spraying system that is used for herbicide treatment. We expect active enzyme can be available in the cells within a shorter period by protein delivery than nucleic acids delivery, therefore, protein delivery is suitable especially for insect treatment application which requires quick treatment. Proteins are mixed with protease inhibitors to minimize the protein degradation, and then mixed with CPP. After 24 hours of application, the herbicide chemical for corresponding enzymes is sprayed in the field using the sprayer. We select CPPs which penetrate efficiently soybean leaf but not leaves of weeds like morning glory. The soybean plant transiently gains the tolerance to the herbicide and then sequential herbicide spraying successfully kills only weeds around soybean. This method can be automated. Weeds can be detected and captured by drone camera, and weed intensity can be analyzed by image analysis program, and semi-automatic application of the CPP-biomolecule solution, followed by spraying of herbicide solution can reduce weed presence.

We here demonstrate the agriculture large-scale CPP-biomolecule complexes spray application using DNA, RNA as a biomolecule in soybean as an example. This approach is valuable not only for row crops, but also for vegetables where transgenic plants are not yet accepted by public, such as pepper, onion and carrot.

### References

1. Sambrook, J., E. F. Fritsch, and T. Maniatis (1989) Molecular cloning: a laboratory manual, 2nd ed. Cold Spring Harbor Laboratory Press.

2. F. Sanger, S. Nicklen, and A. R. Coulson (1977) DNA sequencing with chain-terminating inhibitors. PNAS 74(12):5463-5467.

3. Altschul S F, Gish W, Miller W, Myers E W, Lipman D J (1990) Basic local alignment search tool. J Mol Biol. 215(3):403-410.

4. Altschul S F, Madden T L, Schaffer A A, et al. (1997) Gapped BLAST and PSI-BLAST: a new generation of protein database search programs. Nucleic Acids Res. 25(17):3389-3402.

5. J. F. Sambrook and D. W. Russell, ed. Molecular Cloning: A Laboratory Manual, 3rd ed. (2001) Vols 1,2 and 3, Cold Spring Harbor Laboratory Press.

6. Dalakouras A, et al. (2020) Genetically Modified Organism-Free RNA Interference: Exogenous Application of RNA Molecules in Plants. Plant Physiol 182(1):38-50.

7. Dalakouras A, Wassenegger M, McMillan J N, Cardoza V, Maegele I, Dadami E, Runne M, Krczal G, Wassenegger M (2016) Induction of Silencing in Plants by High-Pressure Spraying of In Vitro-Synthesized Small RNAs. Front Plant Sci 7:1327.

8. Numata K, Ohtani M, Yoshizumi T, Demura T, Kodama Y (2014) Local Gene Silencing in Plants via Synthetic DsRNA and Carrier Peptide. Plant Biotechnol J 12(8):1027-1034.

9. Zhang H, Zhang H, Demirer G S, Gonzalez-Grandio E, Fan C, Landry M P (2020) Engineering DNA Nanostructures for SiRNA Delivery in Plants. Nat Protoc. 15(9):3064-3087.

10. Demirer G S, Zhang H, Goh N S, Pinals R L, Chang R, Landry M P Carbon (2020) Nanocarriers Deliver SiRNA to Intact Plant Cells for Efficient Gene Knockdown. Sci Adv 6(26):eaaz0495.

11. Kim D H, Behlk M A, Rose S D, Chang M S, Choi S, Rossi J J (2005) Synthetic DsRNA Dicer Substrates Enhance RNAi Potency and Efficacy. Nat. Biotechnol 23(2):222-226.

12. Schwartz S H, Hendrix B, Hoffer P, Sanders R A, Zheng W (2020) Carbon Dots for Efficient Small Interfering RNA Delivery and Gene Silencing in Plants. Plant Physiol 184 (2):647-657.

Sequences

SEQ ID No: 1, GUS cDNA sequence
ATGGGGTTACGTCCTGTAGAAACCCCAACCCGTGAAATCAAAAAACTCGACGGCCTGTGGGCATTCAGTCTGG
ATAGGGAGAACTGTGGAATCGACCAACGTTGGTGGGAAAGCGCGTTACAAGAAAGCCGGGCTATCGCTGTG
CCAGGCAGTTTTAACGATCAGTTCGCCGATGCAGATATTCGTAATTATGCGGGCAACGTCTGGTATCAGCGCG
AAGTCTTTATACCGAAAGGTTGGGCAGGCCAGCGTATCGTGCTGCGTTTCGATGCGGTCACTCATTACGGCAA
AGTGTGGGTCAATAATCAGGAAGTGATGGAGCATCAGGGCGGCTATACGCCATTTGAAGCCGATGTCACGCC
GTATGTTATTGCCGGGAAAAGTCTACGTAAGTTTCTGCTTCTACCTTTGATATATATATAATAATTATCATTAAT
TAGTAGTAATATAATATTTCAAATATTTTTTTCAAAATAAAAGAATGTAGTATATAGCAATTCCTTTTCTGTAGT
TTATAAGTGTGTATATTTTAATTTATAACTTTTCTAATATATGACCAAAATTTGTTGATGTGCAGGTATCACCGT
TTGTGTGAACAACGAACTGAACTGGCAGACTATCCCGCCGGGAATGGTGATTACCGACGAAAACGGCAAGAA
AAAGCAGTCTTACTTCCATGATTTCTTTAACTATGCCGGAATCCATCGCAGCGTAATGCTCTACACCACGCCGA
ACACCTGGGTGGACGATATAACCGTGGTGACGCATGTCGCGCAAGACTGTAACCACGCTTCTGTTGACTGGCA
AGTTGTGGCCAATGGTGATGTCAGCGTTGAACTGCGTGATGCGGATCAACAGGTGGTTGCAACTGGACAAGG
CACTAGCGGGACTTTGCAAGTGGTGAATCCGCACCTCTGGCAACCGGGTGAAGGTTATCTCTATGAACTGTGC
GTCACAGCCAAAAGCCAGACAGAGTGTGATATTTACCCGCTTCGCGTCGGCATCCGGTCAGTGGCAGTGAAG
GGCGAACAGTTCCTGATTAACCACAAACCGTTCTACTTTACTGGCTTTGGTCGTCATGAAGATGCGGACTTGCG
TGGCAAAGGATTCGATAACGTGCTGATGGTTCACGACCACGCTCTTATGGACTGGATTGGGGCCAACTCCTAC
CGTACCTCGCATTACCCTTACGCTGAAGAAATGCTCGACTGGGCAGATGAACATGGCATCGTGGTGATTGATG
AAACTGCTGCTGTCGGCTTTAACCTCTCTTTAGGCATTGGTTTCGAGGCGGGCAACAAGCCGAAAGAACTGTA
CAGCGAGGAAGCAGTCAACGGGGAAACTCAGCAAGCGCACTTACAGGCGATCAAGGAGCTGATAGCGCGTG
ACAAAAACCACCCAAGCGTGGTGATGTGGAGTATTGCCAACGAACCGGATACCCGTCCGCAAGGAGCTAGGG
AGTATTTCGCGCCACTGGCGGAAGCAACCAGAAAACTCGACCCGACCAGGCCGATCACCTGTGTCAATGTAAT
GTTCTGCGACGCTCACACCGATACCATCAGCGATCTCTTTGATGTGCTGTGCCTGAACCGTTATTACGGATGGT
ATGTCCAAAGCGGCGATTTGGAAACGGCAGAGAAGGTACTGGAAAAAGAACTTCTGGCCTGGCAGGAGAAA
CTGCATCAGCCGATTATCATCACCGAATACGGCGTGGATACGTTAGCCGGGCTGCACTCAATGTACACCGACA
TGTGGAGTGAGGAGTATCAGTGTGCATGGCTGGATATGTATCACCGCGTCTTTGATCGCGTCAGCGCCGTCGT
CGGTGAACAGGTATGGAATTTCGCCGATTTTGCGACCTCGCAAGGCATATTGCGCGTTGGCGGTAACAAGAA
AGGGATCTTCACCAGGGATCGCAAACCGAAGTCGGCGGCTTTTCTGCTGCAAAAACGCTGGACTGGCATGAA
CTTCGGTGAAAAACCGCAGCAGGGAGGCAAACAATGA
SEQ ID No: 2, GFP cDNA sequence
ATGGTGAGCAAGGGCGAGGAGCTGTTCACCGGGGTGGTGCCCATCCTGGTCGAGCTGGACGGCGACGTAAA
CGGCCACAAGTTCAGCGTGTCCGGCGAGGGCGAGGGCGATGCCACCTACGGCAAGCTGACCCTGAAGTTCAT
CTGCACCACCGGCAAGCTGCCCGTGCCCTGGCCCACCCTCGTGACCACCCTGACCTACGGCGTGCAGTGCTTC
AGCCGCTACCCCGACCACATGAAGCAGCACGACTTCTTCAAGTCCGCCATGCCCGAAGGCTACGTCCAGGAGC
GCACCATCTTCTTCAAGGACGACGGCAACTACAAGACCCGCGCCGAGGTGAAGTTCGAGGGCGACACCCTGG
TGAACCGCATCGAGCTGAAGGGCATCGACTTCAAGGAGGACGGCAACATCCTGGGGCACAAGCTGGAGTAC
AACTACAACAGCCACAACGTCTATATCATGGCCGACAAGCAGAAGAACGGCATCAAGGTGAACTTCAAGATC
CGCCACAACATCGAGGACGGCAGCGTGCAGCTCGCCGACCACTACCAGCAGAACACCCCCATCGGCGACGGC
CCCGTGCTGCTGCCCGACAACCACTACCTGAGCACCCAGTCCGCCCTGAGCAAAGACCCCAACGAGAAGCGC
GATCACATGGTCCTGCTGGAGTTCGTGACCGCCGCCGGGATCACTCTCGGCATGGACGAGCTGTACAAGTAA
SEQ ID No: 3, DsRed cDNA sequence
ATGGGGTCTTCCAAGAATGTTATCAAGGAGTTCATGAGGTTTAAGGTTCGCATGGAAGGAACGGTCAATGGG
CACGAGTTTGAAATAGAAGGCGAAGGAGAGGGGAGGCCATACGAAGGCCACAATACCGTAAAGCTTAAGGT
AACCAAGGGGGGACCTTTGCCATTTGCTTGGGATATTTTGTCACCACAATTTCAGTATGGAAGCAAGGTATAT
GTCAAGCACCCTGCCGACATACCAGACTATAAAAAGCTGTCATTTCCTGAAGGATTTAAATGGGAAAGGGTCA
TGAACTTTGAAGACGGTGGCGTCGTTACTGTAACCCAGGATTCCAGTTTGCAGGATGGCTGTTTCATCTACAA
GGTCAAGTTCATTGGCGTGAACTTTCCTTCCGATGGACCTGTTATGCAAAAGAAGACAATGGGCTGGGAAGCC
AGCACTGAGCGTTTGTATCCTCGTGATGGCGTGTTGAAAGGAGAGATTCATAAGGCTCTGAAGCTGAAAGAC
GGTGGTCATTACCTAGTTGAATTCAAAAGTATTTACATGGCAAAGAAGCCTGTGCAGCTACCAGGGTACTACT
ATGTTGACTCCAAACTGGATATAACAAGCCACAACGAAGACTATACAATCGTTGAGCAGTATGAAAGAACCGA
GGGACGCCACCATCTGTTCCTTTAG
SEQ ID No: 4, Renilla luciferase (Rluc) cDNA sequence
ATGGCTTCGAAGGTGTACGACCCCGAGCAGAGGAAGAGGATGATCACCGGCCCCCAGTGGTGGGCCAGGTG
CAAGCAGATGAACGTGCTGGACAGCTTCATCAACTACTACGACAGCGAGAAGCACGCCGAGAACGCCGTGAT
CTTCCTGCACGGCAACGCCGCTAGCAGCTACCTGTGGAGGCACGTGGTGCCCCACATCGAGCCCGTGGCCAG
GTGCATCATCCCCGATCTGATCGGCATGGGCAAGAGCGGCAAGAGCGGCAACGGCAGCTACAGGCTGCTGG
ACCACTACAAGTACCTGACCGCCTGGTTCGAGCTCCTGAACCTGCCCAAGAAGATCATCTTCGTGGGCCACGA
CTGGGGCGCCTGCCTGGCCTTCCACTACAGCTACGAGCACCAGGACAAGATCAAGGCCATCGTGCACGCCGA
GAGCGTGGTGGACGTGATCGAGAGCTGGGACGAGTGGCCAGACATCGAGGAGGACATCGCCCTGATCAAGA
GCGAGGAGGGCGAGAAGATGGTGCTGGAGAACAACTTCTTCGTGGAGACCGTGCTGCCCAGCGTTATCATG
AGAAAGCTGGAGCCCGAGGAGTTCGCCGCCTACCTGGAGCCCTTCAAGGAGAAGGGCGAGGTGAGAAGACC
CACCCTGAGCTGGCCCAGAGAGATCCCCCTGGTGAAGGGCGGCAAGCCCGACGTGGTGCAGATCGTGAGAA
ACTACAACGCCTACCTGAGAGCCAGCGACGACCTGCCCAAGATGTTCATCGAGAGCGACCCCGGCTTCTTCAG
CAACGCCATCATTGAGGGCGCCAAGAAGTTCCCCAACACCGAGTTCGTGAAGGTGAAGGGCCTGCACTTCAG
CCAGGAGGACGCCCCCGACGAGATGGGCAAGTACATCAAGAGCTTCGTGGAGAGAGTGCTGAAGAACGAGC
AGAGATCTATCTAG
SEQ ID No: 5, ALS cDNA sequence
ATGGCGGCGGCAACAACAACAACAACAACATCTTCTTCGATCTCCTTCTCCACCAAACCATCTCCTTCCTCCTCC
AAATCACCATTACCAATCTCCAGATTCTCCCTCCCATTCTCCCTAAACCCCAACAAATCATCCTCCTCCTCCCGCC
GCCGCGGTATCAAATCCAGCTCTCCCTCCTCCATCTCCGCCGTGCTCAACACAACCACCAATGTCACAACCACT
CCCTCTCCAACCAAACCTACCAAACCCGAAACATTCATCTCCCGATTCGCTCCAGATCAACCCCGCAAAGGCGC
TGATATCCTCGTCGAAGCTTTAGAACGTCAAGGCGTAGAAACCGTATTCGCTTACCCTGGAGGTGCATCAATG
GAGATTCACCAAGCCTTAACCCGCTCTTCCTCAATCCGTAACGTCCTTCCTCGTCACGAACAAGGAGGTGTATT
CGCAGCAGAAGGATACGCTCGATCCTCAGGTAAACCAGGTATCTGTATAGCCACTTCAGGTCCCGGAGCTACA
AATCTCGTTAGCGGATTAGCCGATGCGTTGTTAGATAGTGTTCCTCTTGTAGCAATCACAGGACAAGTCCCTCG
TCGTATGATTGGTACAGATGCGTTTCAAGAGACTCCGATTGTTGAGGTAACGCGTTCGATTACGAAGCATAAC
TATCTTGTGATGGATGTTGAAGATATCCCTAGGATTATTGAGGAAGCTTTCTTTTTAGCTACTTCTGGTAGACC
TGGACCTGTTTTGGTTGATGTTCCTAAAGATATTCAACAACAGCTTGCGATTCCTAATTGGGAACAGGCTATGA
GATTACCTGGTTATATGTCTAGGATGCCTAAACCTCCGGAAGATTCTCATTTGGAGCAGATTGTTAGGTTGATT
TCTGAGTCTAAGAAGCCTGTGTTGTATGTTGGTGGTGGTTGTTTGAATTCTAGCGATGAATTGGGTAGGTTTG
TTGAGCTTACGGGGATCCCTGTTGCGAGTACGTTGATGGGGCTGGGATCTTATCCTTGTGATGATGAGTTGTC
GTTACATATGCTTGGAATGCATGGGACTGTGTATGCAAATTACGCTGTGGAGCATAGTGATTTGTTGTTGGCG
TTTGGGGTAAGGTTTGATGATCGTGTCACGGGTAAGCTTGAGGCTTTTGCTAGTAGGGCTAAGATTGTTCATA
TTGATATTGACTCGGCTGAGATTGGGAAGAATAAGACTCCTCATGTGTCTGTGTGTGGTGATGTTAAGCTGGC
TTTGCAAGGGATGAATAAGGTTCTTGAGAACCGAGCGGAGGAGCTTAAGCTTGATTTTGGAGTTTGGAGGAA
TGAGTTGAACGTACAGAAACAGAAGTTTCCGTTGAGCTTTAAGACGTTTGGGGAAGCTATTCCTCCACAGTAT
GCGATTAAGGTCCTTGATGAGTTGACTGATGGAAAAGCCATAATAAGTACTGGTGTCGGGCAACATCAAATG
TGGGCGGCGCAGTTCTACAATTACAAGAAACCAAGGCAGTGGCTATCATCAGGAGGCCTTGGAGCTATGGGA
TTTGGACTTCCTGCTGCGATTGGAGCGTCTGTTGCTAACCCTGATGCGATAGTTGTGGATATTGACGGAGATG
GAAGCTTTATAATGAATGTGCAAGAGCTAGCCACTATTCGTGTAGAGAATCTTCCAGTGAAGGTACTTTTATTA
AACAACCAGCATCTTGGCATGGTTATGCAATGGGAAGATCGGTTCTACAAAGCTAACCGAGCTCACACATTTC
TCGGGGATCCGGCTCAGGAGGACGAGATATTCCCGAACATGTTGCTGTTTGCAGCAGCTTGCGGGATTCCAG
CGGCGAGGGTGACAAAGAAAGCAGATCTCCGAGAAGCTATTCAGACAATGCTGGATACACCAGGACCTTACC
TGTTGGATGTGATTTGTCCGCACCAAGAACATGTGTTGCCGATGATCCCGAATGGTGGCACTTTCAACGATGT
CATAACGGAAGGAGATGGCCGGATTAAATACTGA
SEQ ID No: 6, PPO cDNA sequence
ATGGTTATTCAGTCTATTACCCACCTCTCCCCAAACCTCGCTTTGCCATCTCCACTTTCTGTGTCCACCAAGAACT
ACCCAGTTGCTGTGATGGGCAACATCTCTGAGAGAGAGGAACCTACCTCTGCTAAGAGGGTTGCAGTTGTTG
GAGCTGGTGTTTCTGGACTTGCTGCTGCTTACAAGCTCAAGTCCCACGGACTTTCAGTGACCCTTTTCGAGGCT
GATTCTAGGGCTGGTGGAAAGCTTAAGACCGTGAAGAAGGATGGCTTCATCTGGGATGAGGGTGCTAACACT
ATGACCGAGTCTGAGGCTGAGGTGTCCTCCCTTATTGATGATCTTGGCCTCAGAGAGAAGCAACAGCTCCCAA
TCTCTCAGAACAAGCGTTACATTGCTAGGGATGGACTTCCAGTGCTCCTCCCATCTAACCCAGCTGCTTTGCTC
ACCTCCAACATCCTTTCCGCTAAGTCCAAGCTCCAGATCATGCTCGAACCATTCCTTTGGAGGAAGCACAACGC
TACCGAGCTTTCTGATGAGCACGTTCAAGAGTCTGTGGGCGAGTTCTTCGAGAGGCATTTCGGCAAAGAATTC
GTGGACTACGTGATCGATCCATTCGTTGCTGGAACTTGCGGAGGTGATCCTCAGTCTCTTTCTATGCATCACAC
CTTCCCAGAGGTGTGGAACATCGAGAAGAGGTTCGGATCTGTGTTCGCTGGCCTTATCCAGTCCACCCTCTTG
TCCAAGAAAGAAAAGGGTGGCGAGAACGCCTCCATCAAGAAGCCAAGAGTTAGGGGCTCATTCAGCTTCCAA
GGTGGAATGCAAACCCTCGTGGATACCATGTGCAAGCAGCTTGGAGAGGATGAGCTTAAGTTGCAGTGCGAG
GTGCTCAGCCTTTCCTATAACCAGAAGGGAATCCCATCCCTCGGCAACTGGTCTGTGTCATCTATGTCCAACAA
CACCTCCGAGGACCAGTCTTACGATGCTGTTGTTGTGACCGCCCCAATCCGTAACGTGAAAGAAATGAAGATC
ATGAAGTTCGGCAACCCCTTCTCCCTCGACTTCATTCCAGAGGTTACCTACGTGCCACTCTCCGTGATGATTACC
GCTTTCAAGAAAGACAAGGTGAAGAGGCCACTCGAGGGATTCGGAGTGCTCATTCCTTCTAAAGAGCAGCAC
AACGGACTCAAGACTGAGGGAACCCTCTTCTCCTCTATGATGTTCCCAGATAGGGCCCCTTCCGATATGTGCCT
TTTCACTACTGTTGTGGGCGGCTCTAGGAACAGAAAGCTTGCTAACGCTTCCACCGACGAGCTGAAGCAGATC
GTGTCATCTGATCTTCAGCAGCTTCTCGGAACCGAGGACGAACCATCTTTCGTGAACCACCTCTTCTGGTCCAA
CGCTTTCCCACTTTACGGCCACAACTACGATTCTGTGCTCAGGGCTATCGACAAGATGGAAAAGGATCTCCCC
GGCTTCTTCTACGCTGGAAACCATAAGGGTGGTCTGTCTGTGGGAAAGGCTATGGCTTCTGGATGCAAGGCT
GCTGAGCTTGTGATCTCCTACCTCGACTCTCACATCTACGTGAAGATGGACGAAAAGACCGCCTGA
SEQ ID No: 7, BP100
KKLFKKILKYL
SEQ ID No: 8, R9
RRRRRRRRR
SEQ ID No: 9, D-R9 (synthetic)
rrrrrrrrr
SEQ ID No: 10, KH9
KHKHKHKHKHKHKHKHKH
SEQ ID NO: 11, BP100(KH)9
KKLFKKILKYLKHKHKHKHKHKHKHKHKH
SEQ ID NO: 12, BP100CH7
KKLFKKILKYLHHCRGHTVHSHHHCIR

1. A method of introducing a nucleic acid into a plant cell, said method comprising:
a) applying a solution of one or more nucleic acids, complexed to one or more Cell Penetrating Peptides to a plant, plant organ or plant tissue by spraying, and
b) allowing the one or more nucleic acids to enter said plant cell.

2. The method of claim 1, wherein said plant cell is part of a plant tissue, plant organ, plant epidermis, or any other part of a plant.

3. The method of claim 1, wherein said solution comprising one or more nucleic acids, complexed to Cell Penetrating Peptides is an aqueous solution, optionally a salt solution and/or a buffered solution.

4. The method of claim 3, wherein said solution is devoid of any other additives.

5. The method of claim 1, wherein said nucleic acids include one or more of DNA, RNA, or nucleic acid analogues.

6. The method of claim 1, wherein said Cell Penetrating Peptide is a cationic Cell Penetrating Peptide or an amphiphilic Cell Penetrating Peptide.

7. The method of claim 6, wherein said cationic Cell Penetrating Peptide is one of BP100 (SEQ ID NO: 7), R9 (SEQ ID NO: 8) or D-R9 (SEQ ID NO: 9).

8. The method of claim 6, wherein said amphiphilic Cell Penetrating Peptide is KH9 (SEQ ID NO: 10).

9. The method of claim 1, wherein said Cell Penetrating Peptide is BP100, KH9, R9, D-R9, BP100(KH)9, BP100CH7, KAibA(KH9) and or KAibA(D-R9).

10. The method of claim 1, further comprising adding an organelle targeting peptide to either the nucleic acid prior to addition of the Cell Penetrating Peptides or to the Cell Penetrating Peptide-nucleic acid complex.

11. The method of claim 1, wherein said nucleic acid is conjugated to an organelle targeting sequence.

12. The method of claim 11, wherein said nucleic acid is delivered into an organelle.

13. The method of claim 1, wherein the composition is targeted to a subcellular compartment of a plant cell.

14. The method of claim 1, wherein the Cell Penetrating Peptide acts as an organelle targeting peptide.

15. The method of claim 2, wherein said plant organ is one or more of a leaf, stem, root, or reproductive organ.

16. The method of claim 1, wherein spraying on a leaf is on the adaxial and/or abaxial side of a plant leaf.

17. A method of modulating gene expression in a plant cell, said method comprising:
applying a solution of one or more nucleic acids capable of modulating expression of a gene, complexed to one or more Cell Penetrating Peptides dissolved in water to a plant, plant organ or plant tissue by spraying; and
b) allowing the one or more nucleic acids capable of modulating expression of a gene to enter said plant cell and to modulate gene expression.

18. A method of applying a nucleic acid to a plant, said method comprising:
a. complexing said nucleic acid with a Cell Penetrating Peptide in water;
b. applying the solution containing the nucleic acid complexed with the Cell Penetrating Peptide to a plant by spraying; and
c. allowing the complex to enter into plant cells.

19. The method of claim 17, wherein said water solution is supplemented with salt and/or a buffer.

20. (canceled)

21. (canceled)

22. (canceled)

23. (canceled)

24. (canceled)

25. The method of claim 1, wherein the spraying comprises dripping, nebulizing, atomizing, misting or any other form of application wherein the solution is applied without directly contacting the plant.

26. (canceled)

27. The method of claim 1, wherein the solution is allowed to enter the plant cells for a period of time, followed by consecutive application of additional solution by way of spraying.

28. The method of claim 1, wherein the method comprises application to a field crop for selectively controlling the growth of weeds and the solution further comprises a herbicide or other pesticide.

29. (canceled)

30. The method of claim 1, wherein the ratio of nucleic acid to Cell Penetrating Peptide ranges between 0.5 to 2.0.

31. The method of claim 1, wherein the nucleic acid comprises a CRISPR-Cas guide RNA.

32. The method of claim 1, wherein the complex is applied to a plant concurrently with another transfection method.

33. (canceled)

34. (canceled)
